(19) United States
(12) Patent Application Publication
(10) Pub. No.: US 2023/0076015 A1
(43) Pub. Date: Mar. 9, 2023

(54) HANDHELD CAUTERY DEVICE

(71) Applicant: Surgical Design Innovations, Johnston, IA (US)

(72) Inventors: Troy J. Boffeli, Woodbury, MN (US); Graham Hamilton, Concord, CA (US); Shannon M. Rush, San Jose, CA (US); David Kawalik, Chandler, AZ (US); Michael Lee, Johnston, IA (US); Jordan Grossman, Akron, OH (US); Mark Hardy, Lakewood, OH (US)

(21) Appl. No.: 17/941,438

(22) Filed: Sep. 9, 2022

Related U.S. Application Data
(60) Provisional application No. 63/242,148, filed on Sep. 9, 2021.
(60) Provisional application No. 63/312,929, filed on Feb. 23, 2022.

(51) Int. Cl. A61B 18/14

(52) U.S. Cl. CPC A61B 18/14; A61B 2018/1475; A61B 2018/00595

(57) ABSTRACT

Various handheld cauterization device and/or system embodiments having safety features or components incorporated therein. In certain implementations, the device can be a retractable and deployable safety shield sized to receive any known handheld cauterization device. According to other embodiments, the device can be a handheld cauterization device with a retractable and deployable cauterization tip or a retractable and deployable safety shield.

### CROSS-REFERENCE TO RELATED APPLICATION(S)

This application claims the benefit under 35 U.S.C. § 119(e) to U.S. Provisional Application 63/312,929, filed Feb. 23, 2022 and entitled “Handheld Cautery Device,” and to U.S. Provisional Application 63/242,148, filed Sep. 9, 2021 and entitled “Handheld Cautery Device,” both of which are hereby incorporated herein by reference in their entireties.

### FIELD

The various embodiments herein relate to cauterization devices, and more specifically to handheld cauterization devices used in various surgical procedures.

### BACKGROUND

A handheld electrocautery device is a single use, sterile device that is commonly used in open surgical procedures to cauterize actively bleeding tissue, or ligate (cut) tissue with the intent to cauterize the tissue before bleeding begins. Known cautery device designs share several common features and functions, including a power cord on one end, an activation switch that completes the electrical connection, and a metal tip that conducts heat to the tissue for the purposes described above. In most known devices, the metal tip is permanently exposed, which can cause thermal and non-thermal injuries to the patient. Typically, during use of these devices, the surgeon must place the device in a plastic holster to protect the patient after each use. Such a holster is typically attached to the surgical drapes and isolates a potentially hot or pointed metal tip from both the patient and flammable materials. Other known devices utilize a plastic sleeve or cover on the device that can be slid forward or flipped into place to protect the metal tip from burning or cutting the patient when not in use.

Thermal injuries caused by electrocautery devices are considered “never” events by government agencies, which mean that they should never happen. However, such events continue to happen at an unacceptable frequency despite efforts to modify safe use protocols and educate end users.

One type of thermal injury that can occur is a burn injury as a result of a fire created by one of the known devices. That is, when not protected, the hot metal tip can ignite a fire if in contact with oxygen or other flammable materials. It has been the long standing common practice of surgical teams to set the cautery directly on to the surgical drapes when not in use, and this can cause a fire. Burn injuries can also occur if the cautery activation switch is accidently pressed by a person or object. This action can burn the patient's skin, interfere with the grounding pad, and/or ignite flammable material located in the surgical field.

There are also disadvantages to the use of the holster described above. For example, the holster method is highly user dependent, and surgeons have a longstanding habit of setting the cautery device down on the drapes when focused on the procedure instead of holstering the device. It is also common that the cautery device is accidentally pulled out of the holster due to interference with other cords and/or equipment during the course of the procedure.

There are also disadvantages to the movable plastic sleeve or cover. This method is also highly user dependent, with many surgeons finding the protective cover to be cumbersome to engage, especially given that the device is designed to be used in a one-handed fashion. Movable covers could also be in the way when performing delicate surgical maneuvers and the cover may not prevent inadvertent pressing of the activation switch.

There are also several non-thermal issues with the known devices discussed above. For example, damages or injuries can result from the metal tip being sharply pointed. That is, a sharp metal tip can puncture the sterile drape and/or the patient. Further, the metal tip also carries risk of injury to hospital and facility personnel handling such device and/or tip.

Use of a cautery device also generates smoke as tissue is ligated or cauterized. This smoke is foul smelling and potentially hazardous (carcinogenic) when the fumes are inhaled by the surgical team. The traditional approach is to have an assistant suction the smoke as it is generated, but this is not always consistent or effective, and the assistant may not have a spare hand to hold a suction device. Certain known cautery devices incorporate a smoke collection (suction) system, where a suction tube connects directly to the cautery device. One disadvantage of such a system is that it can be cumbersome to use if the added suction tubing makes the handheld unit thick or bulky. Suction devices also make constant noise during use, which can be annoying and distracting to the surgeon and surgical team.

There is a need in the art for improved handheld cautery devices.

### BRIEF SUMMARY

Discussed herein are various handheld cauterization device embodiments with certain components and/or features that reduce the risks of patient injury and/or fire that exist with known handheld cauterization devices. The various features and/or components include retractable cauterization tips, deployable shields, and/or automatic electrical disconnection features. Other embodiments can include an automatically activated and deactivated smoke evacuation system built into certain device implementations.

In Example 1, a safety shield device for an electrocauterization device comprises an elongate body, a lumen defined within the elongate body, wherein the lumen is sized and shaped to receive a handheld electrocauterization device, an actuation button access opening defined along a side of the elongate body, an actuation button cover protruding axially along the side of the elongate body, wherein the actuation button cover is disposed adjacent to the actuation button access opening, a deployment button slot defined along the side of the elongate body, and a biasing structure operably coupled to the elongate body and the handheld electrocauterization device, wherein the biasing structure is tensioned when the elongate body is in a retracted position such that the biasing structure applies a force to the elongate body urging the elongate body toward a deployed position.

Example 2 relates to the device according to Example 1, further comprising a deployment button disposed through the deployment button slot and attached to the handheld electrocauterization device.

Example 3 relates to the device according to Example 1, further comprising a distal opening defined at a distal end of the elongate body, wherein the distal opening is in fluidic communication with the lumen.

Example 4 relates to the device according to Example 3, wherein a cauterization tip of the handheld electrocauterization device extends out of the distal opening of the elongate body when the elongate body is in the retracted position.

Example 5 relates to the device according to Example 3, wherein a cauterization tip of the handheld electrocauterization device is disposed within the lumen when the elongate body is in the deployed position.

Example 6 relates to the device according to Example 1, wherein the actuation button access opening is disposed adjacent to an actuation button of the handheld electrocauterization device when the elongate body is in the retracted position, whereby the actuation button of the handheld electrocauterization device is accessible by a user through the actuation button access opening.

Example 7 relates to the device according to Example 1, wherein the actuation button cover is disposed over an actuation button of the handheld electrocauterization device when the elongate body is in the deployed position, whereby the actuation button of the handheld electrocauterization device is inaccessible by a user.

In Example 8, a handheld cauterization device comprises a device body, a body lumen defined along a length of the device body, a distal opening defined in a distal end of the device body, wherein the distal opening is in fluidic communication with the body lumen, a deployable safety shield comprising an elongate shield body moveably disposed within the body lumen, the shield body comprising a shield lumen defined therein, an elongate conductor disposed within the shield lumen, the elongate conductor comprising a cauterization tip disposed at a distal end of the elongate conductor such that the cauterization tip extends distally out of the distal opening, and a cauterization actuation button disposed on a side of the device body along a length of the device body, wherein the deployable safety shield is movable between a retracted position and a deployed position.

Example 9 relates to the device according to Example 8, wherein the distal end of the elongate shield body is disposed over the cauterization tip when the safety shield is in the deployed position, and wherein the cauterization tip extends out of the elongate shield body when the safety shield is in the retracted position.

Example 10 relates to the device according to Example 8, further comprising a button cover disposed at or near a proximal end of the elongate shield body, wherein the button cover protrudes axially through a side opening defined in the device body.

Example 11 relates to the device according to Example 10, wherein the button cover is disposed over the actuation button when the safety shield is disposed in the deployed position, whereby the cauterization actuation button is inaccessible to a user.

Example 12 relates to the device according to Example 10, further comprising a biasing structure operably coupled to the deployable safety shield, wherein the biasing structure is tensioned when the safety shield is in the retracted position such that the biasing structure applies a force to the safety shield urging the safety shield toward the deployed position.

Example 13 relates to the device according to Example 8, further comprising an actuation arm coupled at a first end to the device body and coupled at a second end to the deployable safety shield, wherein the actuation arm is moveable along with the deployable safety shield between the retracted position and the deployed position.

Example 14 relates to the device according to Example 13, wherein the actuation arm comprises a first link rotatably coupled to the first end of the device body and a second link rotatably coupled to the deployable safety shield, wherein the second link is rotatably coupled to the first link.

Example 15 relates to the device according to Example 8, wherein the elongate conductor and cauterization tip are stationary in relation to the device body.

In Example 16, a handheld cauterization device comprises a device body, a lumen defined along a length of the device body, a distal opening defined in a distal end of the device body, wherein the distal opening is in fluidic communication with the lumen, an elongate conductor disposed within the lumen, the elongate conductor comprising a cauterization tip disposed at a distal end of the elongate conductor, wherein the elongate conductor and cauterization tip are moveable between a retracted position and a deployed position in which the cauterization tip extends distally out of the distal opening, a conductor deployment button operably coupled to the elongate conductor, a cauterization actuation button disposed along the length of the device body, and an electrical connection structure disposed along a length of the elongate conductor, wherein the electrical connection structure is engageable with the actuation button when the elongate conductor is disposed in the deployed position and is not engageable with the actuation button when the elongate conductor is disposed in the retracted position.

Example 17 relates to the device according to Example 16, wherein the conductor deployment button is operably coupled to a proximal end of the elongate conductor.

Example 18 relates to the device according to Example 16, further comprising a biased arm operably coupled to the elongate conductor, the biased arm comprising a protrusion extending away from the elongate conductor, and a protrusion opening defined along the length of the device body, the protrusion opening sized to receive the protrusion when the elongate conductor is disposed in the deployed position.

Example 19 relates to the device according to Example 16, further comprising an attachment clip attached to the device body, wherein a portion of the attachment clip is disposed adjacent to the protrusion opening.

Example 20 relates to the device according to Example 16, further comprising a biasing structure operably coupled to the elongate conductor, wherein the biasing structure is tensioned when the elongate conductor is in the deployed position such that the biasing structure applies a force to the elongate conductor urging the elongate conductor toward the retracted position.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1 is a cross-sectional side view of a handheld cauterization device, according to one embodiment.

FIG. 2 is an expanded cross-sectional side view of a proximal portion of the handheld cauterization device of FIG. 1, according to one embodiment.

FIG. 3 is an expanded cross-sectional side view of a distal portion of the handheld cauterization device of FIG. 1, according to one embodiment.

FIG. 4 is an expanded cross-sectional view along the length of the handheld cauterization device of FIG. 1, according to one embodiment.

FIG. 5 is another expanded cross-section side view of a distal portion of the handheld cauterization device of FIG. 1, according to one embodiment.

FIG. 6A is a cross-sectional side view of another handheld cauterization device with the cauterization tip in the deployed position, according to a further embodiment.

FIG. 6B is a cross-sectional side view of the handheld cauterization device of FIG. 6A with the cauterization tip in the retracted position, according to one embodiment.

FIG. 7 is a cross-sectional side view of a further handheld cauterization device, according to another embodiment.

FIG. 8A is a cross-sectional side view of another handheld cauterization device with a protective shield in the retracted position, according to a further embodiment.

FIG. 8B is a cross-sectional side view of the handheld cauterization device of FIG. 8A with the protective shield in the deployed position, according to one embodiment.

FIG. 9 is a cross-sectional side view of yet another handheld cauterization device with a protective shield in the retracted (solid lines) and deployed positions (dotted lines), according to a further embodiment.

FIG. 10A is a cross-sectional side view of a further handheld cauterization device with a protective shield in the retracted position, according to another embodiment.

FIG. 10B is a cross-sectional top view of the handheld cauterization device of FIG. 10A, according to one embodiment.

FIG. 10C is a cross-sectional side view of the handheld cauterization device of FIG. 10A with the protective shield in the deployed position, according to another embodiment.

FIG. 10D is a cross-sectional top view of the handheld cauterization device of FIG. 10C, according to one embodiment.

### DETAILED DESCRIPTION

The various handheld cautery device embodiments disclosed or contemplated herein can have a mechanical retractable metal tip that is fully retractable such that it is fully enclosed within a chamber of the device when it is not in use. Other implementations have a deployable shield or housing (instead of a moveable tip) that can be extended or deployed such that the tip is fully enclosed within the shield or housing. In certain alternative implementations having such a retractable tip or deployable housing, the device can also have a disconnection feature in which the electrical circuit to the tip is disrupted when the tip is in its undeployed or enclosed position. Further, according to some alternative embodiments, a smoke suction system can be incorporated into the device and operably coupled to the retractable tip such that the suction system is activated solely when the tip is in its extended or deployed position.

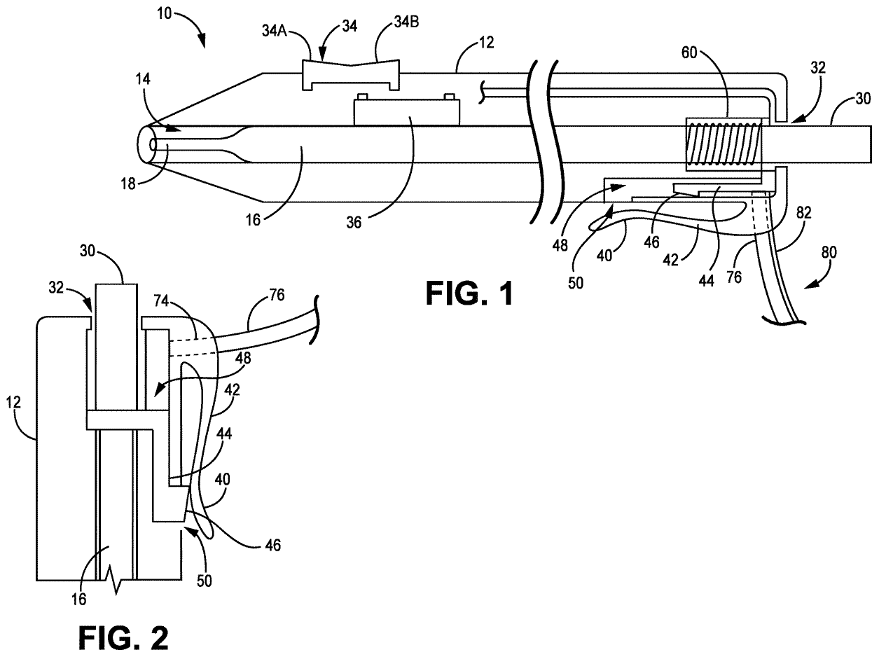

One non-limiting, exemplary embodiment of a cautery device 10 is depicted in FIGS. 1-5. As best shown in FIGS. 1 and 3, the device 10 has a device body 12 with an internal lumen 14 defined therein. Moveably disposed within the lumen 14 is a metal cautery conductor 16 having a cautery tip 18. The conductor 16 can move between a retracted position as shown in FIG. 1 in which the cautery tip 18 is disposed entirely within the lumen 14 and an extended or deployed position as shown in FIG. 3 in which the tip 18 is exposed for use for cauterization of the target tissue of the patient. In the retracted position of FIG. 1, the cautery tip 18 is fully enclosed within the device 10 and thus cannot cause damage to the patient, the user, or any equipment or surrounding objects. In contrast, in the deployed position of FIG. 3, the tip 18 extends from the device body 12 such that it can be used to cauterize any target tissue.

The device body 12 can be made of any known heat stable, non-conductive composite materials commonly used to manufacture medical devices.

As best shown in FIGS. 1 and 2, the device 10 has a conductor deployment (or “actuation”) button 30 moveably disposed at the proximal end of the device body 12 such that the button 30 extends from an opening 32 in the proximal end. The button 30 is operably coupled to the cautery conductor 16 such that the button 30 can be depressed (urged distally) to urge the conductor 16 distally such that the tip 18 is urged into the deployed position of FIG. 3. Given the location of the button 30 and its ease of use, the button 30 allows a user to depress the button 30 and thereby deploy the conductor 16 with one hand.

In addition, according to certain embodiments, the device 10 has a bi-functional cauterization/cutting actuation button 34 actuably disposed on the device body 12 as best shown in FIGS. 1 and 3. The bi-functional button 34 has two actuable portions 34A, 34B on the button such that a user can depress the first or distal portion 34A to actuate cauterization by the cautery tip 18 or the second or proximal portion 34B to actuate a cutting action by the cautery tip 18. Alternatively, the actuation button can be two different buttons, with one for actuation of cauterization and one for actuation of cutting.

In accordance with various implementations, the device 10 can also have an electrical connection panel 36 that is coupled to the cautery conductor 16 such that the panel 36 moves with the conductor 16 between the retracted and deployed positions. Further, the panel 36 is engageable with the bi-functional actuation button 34 when the conductor 16 is in the deployed position, thereby completing the electrical circuit between the bi-functional actuation button 34 and the conductor 16 and allowing for actuation of the cauterization and/or cutting action by the cautery tip 18. Thus, as best shown in FIG. 1, when the conductor 16 is in the retracted position, the panel 36 is not adjacent to the bi-functional actuation button 34 and thus a user cannot actuate cauterization or cutting of any target tissue, thereby preventing any accidental injuries or damage of any kind. On the other hand, as best shown in FIG. 3, when the conductor 16 is in the deployed position, the panel 36 is adjacent to the button 34, such that either of the button portions 34A, 34B can be depressed to cause the relevant portion 34A, 34B to come into contact with the panel 36, thereby completing the electrical circuit and actuating either cauterization or cutting or both.

According to certain embodiments and in any of the various implementations discussed elsewhere herein, the connection panel 36 is also a controller, having a processor disposed therein or associated therewith that can operate to control the cauterization and/or cutting actions of the device. Alternatively, a controller can be provided that is located elsewhere on the device 10 or disposed externally and coupled to the device 10.

In use, the retraction of the conductor 16 into the retracted position disrupts the electrical circuit coupled to the cautery tip 18, thereby making it impossible to inadvertently activate the device 10 if the bi-functional button 34 is accidentally depressed. And once the surgeon or assistant is ready to use the device 10, the user simply needs to depress the conductor actuation button 30 to urge the conductor 16 into the deployed position. This causes the connection panel 36 to also be urged distally until it is in electrical contact with the button 34, thereby completing the electrical circuit and making it possible for the user to actuate the device 10 to cauterize and/or cut a target patient tissue by depressing the actuation button 34 accordingly (as described elsewhere herein).

As best shown in FIGS. 1 and 2, the device 10 also has a retraction button (or arm) 40 disposed at a proximal end of the device 10 that can be depressed to actuate the conductor 16 to retract into the retracted position. Further, the conductor 16 has a biased arm 44 attached to the conductor 16 and disposed within an arm channel 48 such that the arm 44 has a length that is parallel with and extends along a length of the channel 48 adjacent to the conductor lumen 14 as shown. Thus, when the conductor 16 moves along the conductor lumen 14 between its retracted and its deployed positions, the biased arm 44 also moves along the arm channel 48 between retracted and deployed positions. The biased arm 44 is depicted in its retracted position in FIG. 1 and is depicted in its deployed position in FIG. 2. The arm 44 has a protrusion 46 at its distal end that extends radially outward—in a direction away from the conductor 16. The biased arm 44 is biased outwardly such that the protrusion 46 is urged (radially) outwardly by the arm 44. As best shown in FIGS. 1 and 2, the body 12 of the device 10 has an aperture 50 defined in the external wall of the body 12 near the proximal end of the body 12 such that the aperture 50 is in fluidic communication with the arm channel 48. As best shown in FIG. 2, when the conductor 16 is in its deployed position and thus the biased arm 44 is in its deployed position, the protrusion 46 is disposed at the same location in the channel 48 as the aperture 50 such that the biased nature of the arm 44 causes the protrusion 46 to extend out of the channel 48 through the aperture 50.

Once the protrusion 46 is positioned such that it is extending out of the aperture 50, the biased nature of the arm 44 causes the protrusion 46 to remain disposed within the aperture 50 unless and until an external force is applied to the protrusion 46 to overcome the force of the biased arm 44 and thereby urge the protrusion 46 inwardly (toward the conductor 16). That is, the biased arm 44 locks the conductor 16 in the deployed position until the protrusion 46 is urged inwardly such that it is disposed within the channel 48 and no longer in contact with a wall of the aperture 50. At this point, the biased arm 44 and thus the conductor 16 and tip 18 are free to move axially in relation to the device body 12, as discussed in further detail below. In certain embodiments, that force can be provided by the retraction button 40. That is, the aperture 50 is disposed along the length of the device body 12 such that the aperture 50 is in proximity with the distal end of the retraction button 40. As a result, depressing the retraction button 40 can cause the button 40 to come into contact with the protrusion 46, thereby urging it toward the conductor 16 and into the channel 48.

In certain implementations, the device 10 also has a biasing member 60 operably coupled to the conductor 16. More specifically, the biasing member 60 is any biasing structure or mechanism 60 that is coupled to the conductor 16 such that, when the conductor 16 is in its deployed position, the biasing member 60 applies a force to the conductor 16 that urges the conductor 16 in the proximal direction (toward its retracted position). In the specific exemplary embodiment best depicted in FIG. 1, the biasing member 60 is an extension spring 60 that is disposed around the conductor 16 and coupled at its distal end to the conductor 16. Further, the spring 60 is coupled at its proximal end to the device body 12. Thus, the extension spring 60 is disposed in its untensioned state when the conductor 16 is in its retracted position and is urged into a tensioned state when the conductor 16 is urged into its deployed position. As a result, when the user depresses the conductor deployment button 30 to deploy the conductor 16 and tip 18, the user must apply sufficient force to overcome the force of the extension spring 60 being applied to the conductor 16. And when the conductor 16 is urged into its deployed position, the protrusion 46 is urged outwardly such that it extends out of the aperture 50, thereby retaining the biased arm 44 and thus the conductor 16 in the deployed position despite the force being applied to the conductor 16. That is, the biased arm 44 locks the conductor 16 and tip 18 in the deployed position, with the biasing member 60 in its tensioned position. At this point, retraction can only occur if the retraction button 40 is depressed by the user.

When the user wants to retract the cautery tip 18, the user depresses the retraction button 40, which urges the protrusion 46 into the channel 48 until the protrusion 46 is clear of the aperture 50. At this point, the counteracting force of the protrusion 46 being disposed within the aperture 50 has been removed, and the force applied to the conductor 16 by the biasing member 60 urges the conductor 16 and tip 18 proximally into their retracted positions.

Thus, the combination of the biased arm 44, the biasing member 60, and the retraction button 40 provide for a set of mechanisms that make it possible to easily retract the cautery tip 18 into the retracted position by the simply action of depressing a single button—the retraction button 40. In other words, the position of the retraction button 40 on the device 10 and the ease of using the button 40 makes it possible for the user to use one hand to retract the cautery tip 18. As such, actuation of both deployment and retraction can be simple, one-handed processes.

In accordance with certain implementations, the position of the protrusion 46 on the device body 12 can also provide a safety retraction feature when the device 10 is inserted into its holster during a procedure. As best shown in FIG. 2, the retraction button 40 can be disposed at the distal end of an attachment clip 42 attached at its proximal end to the device body 12. In other words, the retraction button 40 IS the distal end of the clip 42. Thus, when the cautery tip 18 is in the deployed position (and the protrusion 46 is disposed in the aperture 50) and the device 10 is inserted into its holster without first depressing the retraction button 40 to retract the tip 18, the positioning of the device 10 in the holster (not shown) such that wall of the holster (not shown) is inserted between the device body 12 and the clip 42 will trigger the safety feature, causing the tip 18 to retract. That is, the insertion of the device 10 into the holster (not shown) such that the holster wall (not shown) is positioned between body 12 and clip 42 will cause the wall (not shown) to urge the protrusion 46 into the channel 48, thereby releasing the biased arm 44 and causing the tip 18 to retract. This safety feature, according to certain embodiments, causes the retraction of the tip 18 when the device 10 is placed in the holster (not shown) even if the user forgets to retract it before placing the device 10 in the holster.

In an alternative embodiment, the conductor deployment button 30 can be a combination deployment/retraction (or “actuation/de-actuation”) button. That is, in such implementations, the button can operate in a fashion similar to the actuation button on a retractable ball-point pen—the button can be depressed to deploy the cautery tip and can be depressed a second time to retract the tip.

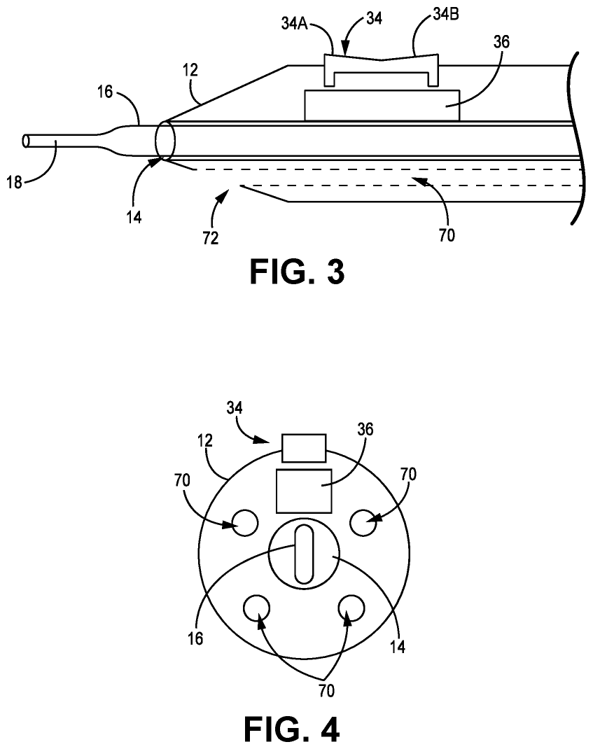

In accordance with certain alternative embodiments, any device 10 implementation herein can also have an automatic smoke evacuation system. As best shown in FIGS. 3-5, the device body 12 can have one or more smoke evacuation lumens 70 defined within the body 12 such that each lumen 70 extends along a substantial portion of the length of the body 12. In the specific embodiment depicted in FIG. 4, the device 10 has four smoke evacuation lumens 70 as shown. As best shown in FIG. 3, each lumen 70 has an opening 72 defined at or near the distal end of the device 10 such that suction applied through the lumen 70 can urge smoke into the lumen 70 through the opening 72. Thus, smoke generated at the cautery tip 18 can be evacuated via the lumens 70.

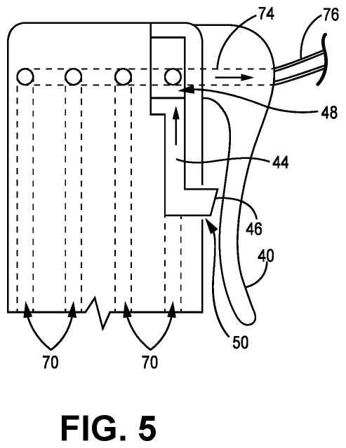

The smoke evacuation system can, in some implementations, be actuable to apply suction solely when the cautery tip 18 is in the deployed position. That is, the device 10 is configured to automatically actuate smoke suction via the evacuation lumen(s) 70 when the conductor 16 is urged into the deployed position by a user, and further to automatically deactivate the smoke suction when the conductor 16 is retracted into the retracted position. This is accomplished, according to one exemplary embodiment, via the configuration depicted in FIG. 5, in which all of the evacuation lumens 70 are in fluid communication with a single proximal transverse lumen 74 in a proximal portion of the device body 12 as shown. That is, each of the two or more smoke evacuation lumens 70 extend from a distal portion of the device body 12 to a proximal portion of the body 12 and are substantially parallel with a longitudinal axis of the body 12. The proximal ends of the lumens 70 are in fluidic communication with the single transverse smoke evacuation lumen 74, which is disposed transversely to the lumens 70 such that the lumen 74 extends axially along a proximal portion of the body 12 and is in fluidic communication with an evacuation tube 76 that extends from the device body 12, as best shown in FIGS. 2 and 5. As such, the external suction source (not shown) creates negative air pressure that causes a suction action in the evacuation tube 76, which thereby causes a suction action in each of the evacuation lumens 70 that causes suction of smoke at the distal end of the device body 12.

In addition, the proximal end of the biased arm 44 can also be in fluidic communication with the transverse lumen 74. That is, the arm channel 48 discussed above is in fluidic communication with the transverse lumen 74, as best shown in FIG. 5. Thus, when the biased arm 46 is disposed in its retracted position within the arm channel 48, the proximal end of the arm 46 is disposed across the transverse lumen 74 such that the arm 46 blocks fluidic communication between the lumen 74 and the tube 76, thereby establishing a fluidic seal that blocks any suction being applied to the lumen 74 via the tube 76. Thus, when the biased arm 44 is in the retracted position blocks fluidic communication between the lumen 74 and the tube 76, there is no suction action. In contrast, when the biased arm 44 is in the deployed position such that the proximal end of the arm 44 is not blocking fluidic communication between the lumen 74 and the tube 76, the suction action of the tube 76 causes suction in the lumen 74 and thus the lumens 70, which causes suction of smoke at the distal end of the device 10. As such, the device 10 only applies suction when the cautery tip 18 is in the deployed position, and once the cautery tip 18 is retracted, the suction is stopped.

This automatic actuation of the smoke evacuation avoids the need to clamp the suction tube when not in use (a common practice with known devices) and eliminates the noise of the suction action by deactivating the suction when the cautery tip 18 is not deployed.

According to certain embodiments, the incorporation of two or more smoke evacuation lumens 70 can result in more successful removal of more smoke. In addition, two or more evacuation lumens 70 result in smoke suction from different angles, thereby improving efficiency.

As best shown in FIG. 1, certain implementations of the device have a cord 80 that extends from a proximal end (or some proximal portion) of the device body 12. The cord 80 is made up of a combination of the smoke evacuation tube 76 discussed above and an electrical cord 82 that couples the conductor 16 with an external electrical source (not shown). Alternatively, the cord 80 can contain any cables or cords that can be coupled to a handheld cautery device.

According to certain embodiments, the cautery device 10 can be a single temperature cautery device, but the various implementations herein can also be variable temperature cauteries. In accordance with some implementations, the device is a single-use device.

In use, the various device embodiments herein can be used to cauterize tissue, make surgical incisions, and/or dissect tissues with the intent to cauterize the tissues that would otherwise bleed upon surgical manipulation. When cauterization is needed, the device 10 is removed from the holster (not shown) as described elsewhere herein. The user then depresses the actuation button 30, thereby actuating the conductor 16 and the cautery tip 18 to move into their deployed position as best shown in FIG. 3, thereby causing two actions to occur. One action is that the electrical connection panel 36 is positioned adjacent to the bi-functional actuation button 34 (as best shown in FIG. 3) such that the actuation button 34 can be depressed to actuate cauterization and/or cutting action. The other action is that the biased arm 44 is urged distally within the arm channel 48, thereby resulting in the transverse evacuation lumen 74 being in fluidic communication with evacuation tube 76 such that suction action is applied to the lumen 74 and the evacuation lumens 70 by the evacuation tube 76, thereby actuating smoke evacuation through the lumens 70, the lumen 74, the tube 76 and into the external suction canister (not shown). Further, the distal movement of the biased arm 44 results in the protrusion 46 reaching and extending out of the aperture 50 as discussed above, thereby locking the cautery tip 18 in place. At this point, with the cautery tip 18 in the deployed position and the smoke evacuation system actuated, the device 10 is ready for use by the surgeon or assistant who simply holds the device 10 in one hand and depresses the desired portion of the bi-functional button 34 to perform cauterization and/or cutting of the desired target tissue.

During use, the device 10 can be intermittently placed in the holster while the conductor 16 and tip 18 are still in the deployed position, especially when using the device 10 frequently such as during the incision and exposure phase of the operation. Alternatively, the conductor 16 and tip 18 can be retracted each time before being placed in the holster.

When the procedure is complete, the surgeon or assistant simply depresses the deployment/retraction (or “actuation/de-actuation”) button 30 to cause the conductor 16 and the tip 18 to be retracted into the retracted position. This retraction causes two actions to occur. One action is that the electrical connection panel 36 moves proximally such that it is no longer positioned adjacent to the bi-functional actuation button 34 (as best shown in FIG. 1), thereby preventing the actuation button 34 from actuating cauterization and/or cutting action when it is depressed. The other action is that the biased arm 44 is urged proximally within the arm channel 48, thereby resulting in the transverse evacuation lumen 74 being blocked from fluidic communication with the evacuation tube 76 and deactivating the smoke evacuation system.

Further, these actions of deployment and retraction can be repeated as often as needed during the course of the procedure.

According to some alternative implementations, any device embodiment herein can also have a safety locking feature that can be triggered by the user when the procedure is complete. That is, once the user has finished the procedure, the user can retract the cautery tip 18 and then depress a button or otherwise actuate the locking feature such that the cautery tip 18 is permanently locked in the retracted position. This locking feature ensures that the cautery tip 18 will not be inadvertently deployed, thereby preventing accidental injuries or damage.

In accordance with certain alternative embodiments, any device implementation herein can also have a safety retraction feature that can be automatically triggered when the user is no longer holding the device. That is, the feature can be configured to actuate the cautery tip 18 to retract into the retracted position whenever the user releases the device. In one embodiment, such automatic actuation can be accomplished with an electrical linear actuator or the like. This automatic retraction feature ensures that the cautery tip 18 is in the retracted position whenever the device is not being held by the user.

One embodiment of the invention is described here but there are a number of design variations that are applicable for specialty applications in surgery or to allow a more cost effective manufacturing process.

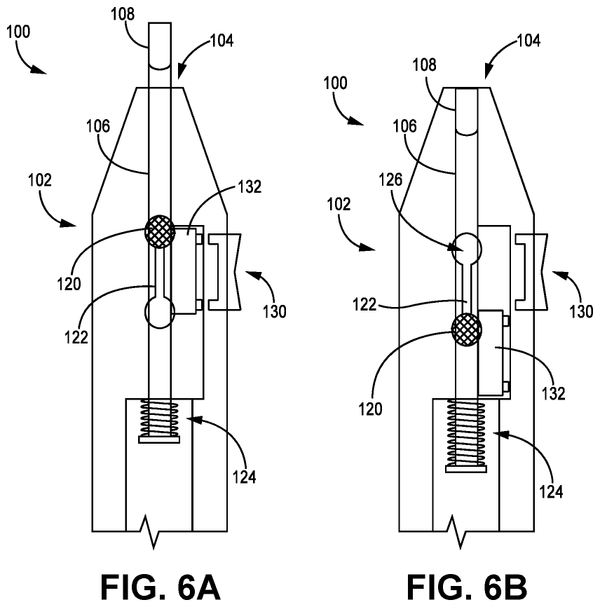

Another exemplary embodiment of a cautery device 100 is depicted in FIGS. 6A and 6B, in which the device 100 is an automatically retracting electrocautery device 100. The device 100 has a device body 102 with an internal lumen 104 defined therein. Moveably disposed within the lumen 104 is a metal cautery conductor 106 having a cautery tip 108. The conductor 106 can move between an extended or deployed position as shown in FIG. 6A in which the tip 108 is exposed for use for cauterization of the target tissue of the patient and a retracted position as shown in FIG. 6B in which the cautery tip 108 is disposed entirely within the lumen 104. The device 100 has a conductor deployment (or “actuation”) button 120 moveably disposed along the side of the device body 102 such that the button 120 extends from a slot 122 defined or disposed in the side of the body 102 as shown, wherein the slot 122 is in fluidic communication with the internal lumen 104. The button 120 is operably coupled to the cautery conductor 106 through the slot 122 such that the button 120 can be urged distally to urge the conductor 106 distally such that the tip 108 is urged into the deployed position of FIG. 6A.

The device 100 also has a biasing member 124 operably coupled to the conductor 106. More specifically, the biasing member 124 can be any biasing structure or mechanism 124 that is coupled to the conductor 106 such that, when the conductor 106 is in its deployed position, the biasing member 124 applies a force to the conductor 106 that urges the conductor 106 in the proximal direction (toward its retracted position). In this specific exemplary embodiment, the biasing member 124 is an compression spring 124 that is disposed around the conductor 106 and coupled at its proximal end to the conductor 106 and coupled at its distal end to the device body 102 as shown. Thus, the compression spring 124 is disposed in its untensioned state when the conductor 106 is in its retracted position and is urged into a tensioned state when the conductor 106 is urged into its deployed position. As a result, when the user urges the conductor deployment button 120 distally to deploy the conductor 106 and tip 108, the user must apply sufficient force to overcome the force of the compression spring 124 being applied to the conductor 106.

The device 100 has no mechanism to lock or otherwise hold the conductor 106 in its deployed position, so the user must continue to apply the necessary amount of force to the button 120 in order to maintain the conductor 106 in its deployed position. Thus, when the user releases or otherwise applies less force to the button 120, the spring 124 causes the conductor 106 to automatically return to its undeployed position. That is, in this exemplary device embodiment, the deployable tip 108 automatically retracts when the device 100 is not in use.

In certain alternative embodiments, the slot 122 can have an opening (or a section having a greater width in comparison to the rest of the slot 122) 126 at a distal end of the slot 122 as shown. Alternatively, the distal end of the slot 122 can be disposed within an indentation 126 defined in the outer surface of the body 102. In one embodiment, when the button 120 is urged to the distal end of the slot 122, the button 120 is received within the opening or indentation 126 such that the button 120 is urged by the force applied by the user down into the opening or indentation 126, thereby providing some temporary retention of the button 120 within the opening or indentation 126 so long as the user continues to apply some force to the button 120. As such, the opening or indentation 126 makes it possible for the user to apply less force to the button 120 once it is positioned within the opening/indentation 126, thereby making it easier for the user to retain the button 120 in the distal position in the slot 122. However, once the user releases or applies less force, the spring 124 urges the button 120 out of the opening/indentation 126 and proximally to the proximal end of the slot 122, thereby urging the conductor 106 and tip 108 into the retracted position.

The remaining components and other features of the device 100 can be substantially similar to the corresponding components and features discussed elsewhere with respect to other embodiments. For example, the device 100 can have a bi-functional cauterization/cutting actuation button 130 and an electrical connection panel 132 that can be substantially similar to and operate in a fashion similar to the button 34 and panel 36 discussed above.

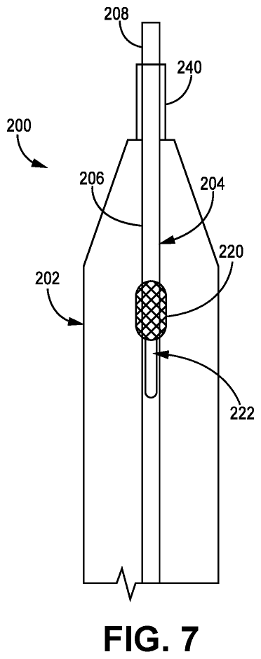

FIG. 7 depicts another implementation of an automatically retracting electrocautery device 200. In this embodiment, the various components and features of the device 200 can be identical or substantially similar to the components and features of the device 100 discussed in detail above, except as discussed herein. More specifically, the device 200 has a tip sheath or cover 240 that extends from the distal end of the body 202 as shown. Thus, the conductor 206 and tip 208 need not retract proximally so far that the tip 208 is disposed within the lumen 204 of the body 202. Instead, the conductor 206 and tip 208 can be retracted proximally only the distance required for the tip 208 to be disposed within the tip sheath 240, thereby resulting in the tip 208 not being capable of making contact with any objects or the patient and thereby causing damage. Thus, the tip 208 can be positioned such that it extends distally out of the lumen 204 and yet, so long as it is disposed within the sheath 240, it is still in an undeployed position. In certain embodiments, this can improve deployment efficiency of the device 200.

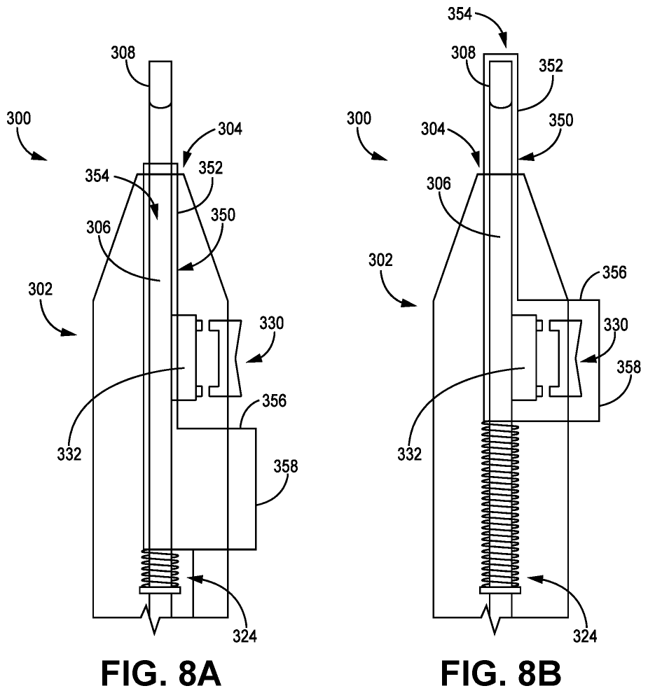

A further embodiment of a cautery device 300 is depicted in FIGS. 8A and 8B, in which the device 300 has an automatically extending safety shield 350. The device 300 has a device body 302 with an internal lumen 304 defined therein. A metal cautery conductor 306 having a cautery tip 308 is fixedly disposed within the lumen 304. That is, the conductor 306 and tip 308 are stationary in relation to the device 302, unlike previous embodiments discussed above. In addition, the device 300 has a bi-functional cauterization/cutting actuation button 330 and an electrical connection panel 332 that can be substantially similar to and operate in a fashion similar to the buttons 34, 130 and panels 36, 132 discussed above. However, unlike the panel 36, 132 versions described above, the electrical connection panel 332 in this embodiment does not move in relation to the device body 302. Instead, because it is attached to the conductor 306, which is stationary in this embodiment, the connection panel 332 is also stationary in relation to the device body 302. Thus, the connection panel 332 in this implementation remains adjacent to the actuation button 330, which operates in the same fashion as the other actuation button embodiments herein to selectively actuate the panel 332 and thus the device 300 in a similar fashion to the selective actuation discussed elsewhere herein.

In this implementation, the device 300 has an extendable shield 350 that is moveably disposed within the lumen 304 and around the conductor 306 and tip 308. More specifically, the shield 350 has an elongate tubular component 352 with a lumen 354 defined therein such that the conductor 306 is disposed within the lumen 354. The shield 350 moves between a deployed or extended position as shown in FIG. 8B and a retracted position as shown in FIG. 8A. In the deployed position, the distal end of the tubular component 352 extends out of the lumen 304 and is disposed around the tip 308 such that the tip 308 is disposed entirely within the lumen 354 of the shield 350. In contrast, in the retracted position, the distal end of the tubular component 352 is disposed within the lumen 304 such that the tip 308 is exposed for use for cauterization of the target tissue of the patient.

The device 300 also has a biasing member 324 operably coupled to the shield 350. More specifically, the biasing member 324 can be any biasing structure or mechanism 324 that is coupled to the shield 350 such that, when the shield 350 is in its retracted position (as shown in FIG. 8A), the biasing member 324 applies a force to the shield 350 that urges the shield 350 in the distal direction (toward its deployed position). In this specific exemplary embodiment, the biasing member 324 is an compression spring 324 that is attached at its distal end with a proximal end of the shield 350 and coupled at its proximal end to the device 300 as shown. Thus, the compression spring 324 is disposed in its untensioned state when the shield 350 is in its deployed position and is urged into a tensioned state when the shield 350 is urged into its retracted position. As a result, when the user urges the shield proximally to retract the shield 350 (and thereby expose the tip 308), the user must apply sufficient force to overcome the force of the compression spring 324 being applied to the shield 350.

The device 300 has no mechanism to lock or otherwise hold the shield 350 in its retracted position, so the user must continue to apply the necessary amount of force to the shield 350 in order to maintain the shield 350 in its deployed position. Thus, when the user releases or otherwise applies less force to the shield 350, the spring 324 causes the shield 350 to automatically return to its deployed position (such that the tip 308 is enclosed). That is, in this exemplary device embodiment, the deployable shield 350 automatically deploys to cover the tip 308 when the device 300 is not in use.

The shield 350 has a shield deployment (or “actuation”) button 356 moveably disposed along the side of the device body 302 such that the button 356 extends from a slot or opening (not shown) defined or disposed in the side of the body 302 as shown, wherein the slot or opening is in fluidic communication with the internal lumen 304. The button 356 is disposed at or near a proximal end of the tubular component 352 and extends through the slot or opening (not shown) such that the button 356 can be urged proximally to urge the tubular component 352 proximally such that the distal end of the tubular component 352 is urged into the retracted position of FIG. 8A such that the tip 308 is fully exposed for use in cauterizing or cutting a target tissue. In one embodiment, the button 356 is actually the distal end of the button cover 358 as discussed below. Alternatively, a button or other protrusion 356 can be incorporated into a proximal portion of the shield 350.

In one alternative embodiment, the shield 350 can also have a button cover 358 as shown in this exemplary version. The button cover 358 is disposed in a proximal portion of the shield and integral with or connected to the proximal end of the tubular component 352. The button cover 358 moves distally and proximally with the shield 350 such that when the shield 350 is in the deployed position, the button cover 358 is disposed over the actuation button 330 such that the button 330 is enclosed within the cover 358 and thus cannot be accidentally actuated. When the shield 350 is in the retracted position, the button cover 358 is moved proximally such that the actuation button 330 is exposed and accessible to the user for actuating the device 300 to cauterize and/or cut the target tissue.

In certain embodiments, including the exemplary embodiment depicted (and as mentioned above), the shield button 356 is the distal end of the button cover 358. Alternatively, the shield button 356 and the button cover 358 can be separate components. In a further alternative, the shield 350 can have no button cover 358.

The remaining components and other features of the device 300 can be substantially similar to the corresponding components and features discussed elsewhere with respect to other embodiments.

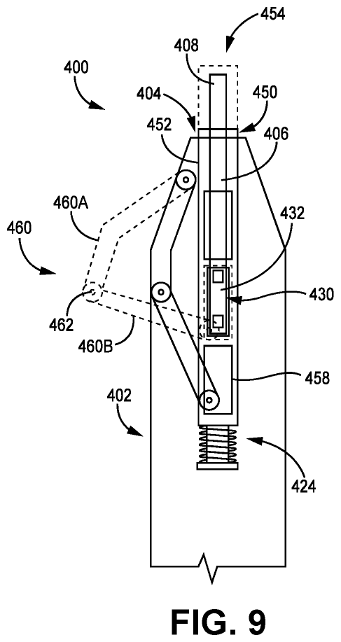

Yet another implementation of a cautery device 400 is depicted in FIG. 9, in which the device 400 has another version of an automatically extending safety shield 450. The device 400 has a device body 402 with an internal lumen 404 defined therein. A metal cautery conductor 406 having a cautery tip 408 is fixedly disposed within the lumen 404. That is, the conductor 406 and tip 408 are stationary in relation to the device 400, unlike some previous embodiments discussed above. In addition, the device 400 has a bi-functional cauterization/cutting actuation button 430 and an electrical connection panel 432 that can be substantially similar to and operate in a fashion similar to the buttons 34, 130 and panels 36, 132 discussed above. However, unlike the panel 36, 132 versions described above (but similar to the panel 332), the electrical connection panel 432 in this embodiment does not move in relation to the device body 402. Instead, because it is attached to the conductor 406, which is stationary in this embodiment, the connection panel 432 is also stationary in relation to the device body 402. Thus, the connection panel 432 in this implementation remains adjacent to the actuation button 430, which operates in the same fashion as the other actuation button embodiments herein to selectively actuate the panel 432 and thus the device 400 in a similar fashion to the selective actuation discussed elsewhere herein.

Like the device 300 discussed above, in this implementation, the device 400 has an extendable shield 450 that is moveably disposed within the lumen 404 and around the conductor 406 and tip 408. More specifically, the shield 450 has an elongate tubular component 452 with a lumen 454 defined therein such that the conductor 406 is disposed within the lumen 454. The shield 450 moves between a deployed or extended position as shown in FIG. 9 in dotted lines and a retracted position as shown in FIG. 9 in solid lines. In the deployed position, the distal end of the tubular component 452 extends out of the lumen 404 and is disposed around the tip 408 such that the tip 408 is disposed entirely within the lumen 454 of the shield 450. In contrast, in the retracted position, the distal end of the tubular component 452 is disposed within the lumen 404 such that the tip 408 is exposed for use for cauterization of the target tissue of the patient.

The device 400 also has a biasing member 424 operably coupled to the shield 450. More specifically, the biasing member 424 can be any biasing structure or mechanism 424 that is coupled to the shield 450 such that, when the shield 450 is in its retracted position (as shown in solid lines), the biasing member 424 applies a force to the shield 450 that urges the shield 450 in the distal direction (toward its deployed position—as shown in dotted lines). In this specific exemplary embodiment, the biasing member 424 is an compression spring 424 that is attached at its distal end with a proximal end of the shield 450 and coupled at its proximal end to the device 400 as shown. Thus, the compression spring 424 is disposed in its untensioned state when the shield 450 is in its deployed position and is urged into a tensioned state when the shield 450 is urged into its retracted position. As a result, when the user urges the shield proximally to retract the shield 450 (and thereby expose the tip 408), the user must apply sufficient force to overcome the force of the compression spring 424 being applied to the shield 450.

In this implementation, the device 400 has an actuation arm 460 having a first link 460A and a second link 460B coupled to the first link 460A at a joint 462. The distal end of the first link 460A is rotatably coupled to the body 402 while the proximal end of the second link 460B is rotatably coupled to the proximal end of the shield 450. The arm 460 is shown in the deployed position in dotted lines (that is, the shield 450 in its deployed position is represented by the dotted lines). The arm 460 can be urged by a user into the retracted position (solid lines) and thereby urge the shield 450 into the retracted position by applying force to the first link 460A (typically via a user's finger or thumb). More specifically, the user can apply an inward radial force (toward the device body 402) to the first link 460A.

The device 400 has no mechanism to lock or otherwise hold the shield 450 in its retracted position, so the user must continue to apply the necessary amount of force to the arm 460 in order to maintain the shield 450 in its retracted position. Thus, when the user releases or otherwise applies less force to the arm 432, the spring 424 causes the shield 450 to automatically return to its deployed position (such that the tip 408 is enclosed). That is, in this exemplary device embodiment, the deployable shield 450 automatically deploys to cover the tip 408 when the device 400 is not in use.

In one alternative embodiment, the shield 450 can also have a button cover 458 as shown in this exemplary version. The button cover 458 is disposed in a proximal portion of the shield 450 and integral with or connected to the proximal end of the tubular component 452. The button cover 458 moves distally and proximally with the shield 450 such that when the shield 450 is in the deployed position, the button cover 458 is disposed over the actuation button 430 (as shown in dotted lines) such that the button 430 is enclosed within the cover 458 and thus cannot be accidentally actuated. When the shield 450 is in the retracted position (as shown in solid lines), the button cover 458 is moved proximally such that the actuation button 430 is exposed and accessible to the user for actuating the device 400 to cauterize and/or cut the target tissue.

The remaining components and other features of the device 400 can be substantially similar to the corresponding components and features discussed elsewhere with respect to other embodiments.

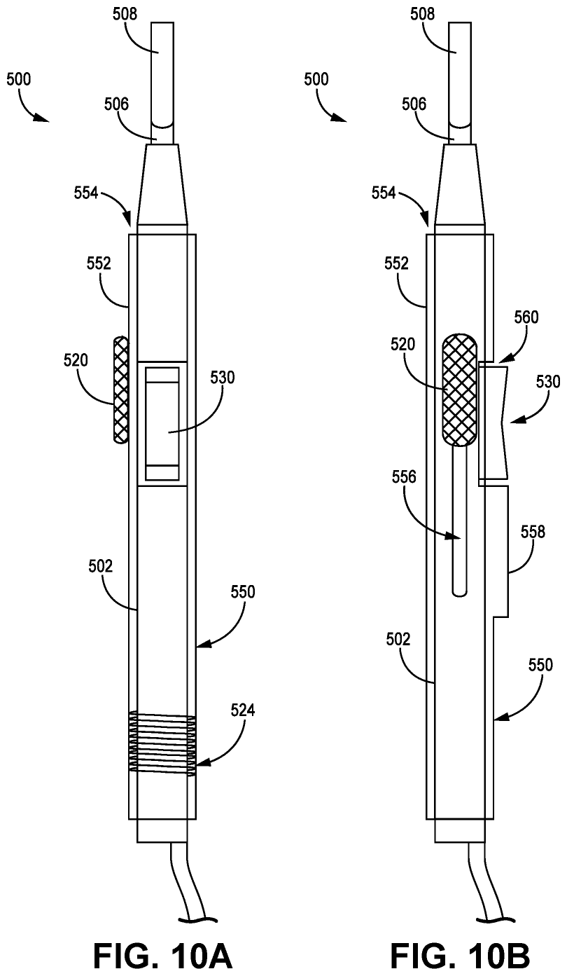

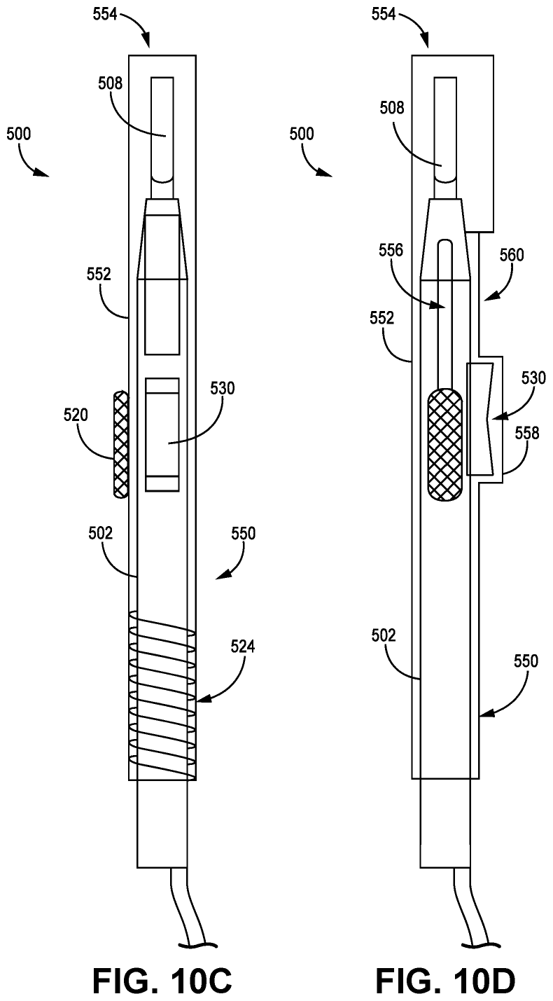

A further embodiment of a cautery device 500 is depicted in FIGS. 10A-10D, in which the device 500 is moveably disposed within a safety shield 550. In certain alternative embodiments, the shield 550 can be provided as a separate component that can be used with any commercially available cauterization device. In those implementations in which a device is provided with the shield 550, the device 500 can have a device body 502 with a metal cautery conductor 506 having a cautery tip 508 extending from the body 502. That is, the conductor 506 and tip 508 are stationary in relation to the device 500. In addition, the device 500 has a bi-functional cauterization/cutting actuation button 530. As mentioned above, the device 500 can operate in a fashion similar to commercially-available devices and can, in some embodiments, be a commercially-available device.

This implementation has an extendable shield 550 that is moveably disposed over the device 500. More specifically, the shield 550 has an elongate body 552 having a lumen 554 defined within the body 552 such that the device 500 can be moveably disposed therein. The shield 550 has a deployment (or “actuation”) button 520 moveably disposed along the side of the body 552 such that the button 520 extends from a slot 556 defined or disposed in the side of the body 552 as shown, wherein the slot 556 is in fluidic communication with the internal lumen 554. The button 520 is operably coupled to the device body 502 through the slot 556 such that the button 520 can be urged distally to urge the device body 502 distally such that the tip 508 is urged into the deployed position of FIGS. 10A and 10B, thereby exposing the tip 508 such that the tip 508 is accessible to cauterize and/or cut the target tissue.

The device 500 also has a biasing member 524 operably coupled to the shield 550. More specifically, the biasing member 524 can be any biasing structure or mechanism 524 that is coupled to the shield 550 and device 500 such that, when the device 500 is in its deployed position (as shown in FIGS. 10A and 10B), the biasing member 524 applies a force to the device 500 that urges the device 500 in the proximal direction (toward its retracted position as shown in FIGS. 10C and 10D). In this specific exemplary embodiment, the biasing member 524 is a compression spring 524 that is attached at its distal end to the shield 550 and coupled at its proximal end to the device 500 as shown. Thus, the compression spring 524 is disposed in its untensioned state when the device 500 is in its retracted position (FIGS. 10C and 10D) and is urged into a tensioned state when the device 500 is urged into its deployed position (FIGS. 10A and 10D). As a result, when the user urges the button 520 distally to urge the device 500 into its deployed position (and thereby expose the tip 508), the user must apply sufficient force to overcome the force of the compression spring 524 being applied to the device 500.

Alternatively, instead of a button, the shield 550 can have an actuation arm similar to the actuation arm 460.

In one alternative embodiment, the shield 550 can also have a button cover 558 and an opening 560 defined in a side of the shield 550 distal of the cover 558 as shown in this exemplary version. The button cover 558 is disposed on and integral with or connected to a side of the shield 550. The button cover 558 moves distally and proximally with the shield 550 such that when the device 500 is in the retracted position, the button cover 558 is disposed over the actuation button 530 such that the button 530 is enclosed within the cover 558 and thus cannot be accidentally actuated. When the device 500 is in the deployed position, the device 500 (and thus the button 530) is moved distally such that the actuation button 530 is exposed in the opening 560 and accessible to the user for actuating the device 500 to cauterize and/or cut the target tissue.

The remaining components and other features of the device 500 can be substantially similar to the corresponding components and features discussed elsewhere with respect to other embodiments.

As mentioned above, the shield 550 can be provided as a separate component that can be used with any commercially available cauterization device. In certain embodiments, the body 552 can be made of two separate, coupleable parts that can be coupled together such that the commercial cauterization device can be inserted into one of the parts and then the second part can be coupled to the first part, thereby enclosing the device within the shield 550. In some implementations, the shield 550 can be designed with a generic shape that will receive many or all commercial devices. Alternatively, the shield 550 can have a specific shape or configuration to be used with any specific commercial cauterization device.

While the various systems described above are separate implementations, any of the individual components, mechanisms, or devices, and related features and functionality, within the various device or system embodiments described in detail above can be incorporated into any of the other embodiments herein.

The term “about,” as used herein, refers to variation in the numerical quantity that can occur, for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The term “about” also encompasses these variations. The term “about” can include any variation of 5% or 10%, or any amount—including any integer—between 0% and 10%. Further, whether or not modified by the term “about,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1%, and 4¾ This applies regardless of the breadth of the range.

Although the various embodiments have been described with reference to preferred implementations, persons skilled in the art will recognize that changes may be made in form and detail without departing from the spirit and scope thereof.

What is claimed is:

1. A safety shield device for an electrocauterization device, the safety shield device comprising:
(a) an elongate body;
(b) a lumen defined within the elongate body, wherein the lumen is sized and shaped to receive a handheld electrocauterization device;
(c) an actuation button access opening defined along a side of the elongate body;
(d) an actuation button cover protruding axially along the side of the elongate body, wherein the actuation button cover is disposed adjacent to the actuation button access opening;
(e) a deployment button slot defined along the side of the elongate body; and
(f) a biasing structure operably coupled to the elongate body and the handheld electrocauterization device, wherein the biasing structure is tensioned when the elongate body is in a retracted position such that the biasing structure applies a force to the elongate body urging the elongate body toward a deployed position.

2. The safety shield device of claim 1, further comprising a deployment button disposed through the deployment button slot and attached to the handheld electrocauterization device.

3. The safety shield device of claim 1, further comprising a distal opening defined at a distal end of the elongate body, wherein the distal opening is in fluidic communication with the lumen.

4. The safety shield device of claim 3, wherein a cauterization tip of the handheld electrocauterization device extends out of the distal opening of the elongate body when the elongate body is in the retracted position.

5. The safety shield device of claim 3, wherein a cauterization tip of the handheld electrocauterization device is disposed within the lumen when the elongate body is in the deployed position.

6. The safety shield device of claim 1, wherein the actuation button access opening is disposed adjacent to an actuation button of the handheld electrocauterization device when the elongate body is in the retracted position, whereby the actuation button of the handheld electrocauterization device is accessible by a user through the actuation button access opening.

7. The safety shield device of claim 1, wherein the actuation button cover is disposed over an actuation button of the handheld electrocauterization device when the elongate body is in the deployed position, whereby the actuation button of the handheld electrocauterization device is inaccessible by a user.

8. A handheld cauterization device comprising:
(a) a device body;
(b) a body lumen defined along a length of the device body;
(c) a distal opening defined in a distal end of the device body, wherein the distal opening is in fluidic communication with the body lumen;
(d) a deployable safety shield comprising an elongate shield body moveably disposed within the body lumen, the shield body comprising a shield lumen defined therein;
(e) an elongate conductor disposed within the shield lumen, the elongate conductor comprising a cauterization tip disposed at a distal end of the elongate conductor such that the cauterization tip extends distally out of the distal opening; and
(f) a cauterization actuation button disposed on a side of the device body along a length of the device body,
wherein the deployable safety shield is movable between a retracted position and a deployed position.

9. The handheld cauterization device of claim 8, wherein the distal end of the elongate shield body is disposed over the cauterization tip when the safety shield is in the deployed position, and wherein the cauterization tip extends out of the elongate shield body when the safety shield is in the retracted position.

10. The handheld cauterization device of claim 8, further comprising a button cover disposed at or near a proximal end of the elongate shield body, wherein the button cover protrudes axially through a side opening defined in the device body.

11. The handheld cauterization device of claim 10, wherein the button cover is disposed over the actuation button when the safety shield is disposed in the deployed position, whereby the cauterization actuation button is inaccessible to a user.

12. The handheld cauterization device of claim 10, further comprising a biasing structure operably coupled to the deployable safety shield, wherein the biasing structure is tensioned when the safety shield is in the retracted position such that the biasing structure applies a force to the safety shield urging the safety shield toward the deployed position.

13. The handheld cauterization device of claim 8, further comprising an actuation arm coupled at a first end to the device body and coupled at a second end to the deployable safety shield, wherein the actuation arm is moveable along with the deployable safety shield between the retracted position and the deployed position.

14. The handheld cauterization device of claim 13, wherein the actuation arm comprises:
(a) a first link rotatably coupled to the first end of the device body; and
(b) a second link rotatably coupled to the deployable safety shield,
wherein the second link is rotatably coupled to the first link.

15. The handheld cauterization device of claim 8, wherein the elongate conductor and cauterization tip are stationary in relation to the device body.

16. A handheld cauterization device comprising:
(a) a device body;
(b) a lumen defined along a length of the device body;
(c) a distal opening defined in a distal end of the device body, wherein the distal opening is in fluidic communication with the lumen
(d) an elongate conductor disposed within the lumen, the elongate conductor comprising a cauterization tip disposed at a distal end of the elongate conductor, wherein the elongate conductor and cauterization tip are moveable between a retracted position and a deployed position in which the cauterization tip extends distally out of the distal opening;
(e) a conductor deployment button operably coupled to the elongate conductor;
(f) a cauterization actuation button disposed along the length of the device body; and
(g) an electrical connection structure disposed along a length of the elongate conductor, wherein the electrical connection structure is engageable with the actuation button when the elongate conductor is disposed in the deployed position and is not engageable with the actuation button when the elongate conductor is disposed in the retracted position.

17. The handheld cauterization device of claim 16, wherein the conductor deployment button is operably coupled to a proximal end of the elongate conductor.

18. The handheld cauterization device of claim 16, further comprising:
(a) a biased arm operably coupled to the elongate conductor, the biased arm comprising a protrusion extending away from the elongate conductor; and
(b) a protrusion opening defined along the length of the device body, the protrusion opening sized to receive the protrusion when the elongate conductor is disposed in the deployed position.

19. The handheld cauterization device of claim 16, further comprising an attachment clip attached to the device body, wherein a portion of the attachment clip is disposed adjacent to the protrusion opening.

20. The handheld cauterization device of claim 16, further comprising a biasing structure operably coupled to the elongate conductor, wherein the biasing structure is tensioned when the elongate conductor is in the deployed position such that the biasing structure applies a force to the elongate conductor urging the elongate conductor toward the retracted position.
